(19) United States
(12) Patent Application Publication
(10) Pub. No.: US 2008/0119723 A1
(43) Pub. Date: May 22, 2008

(54) Localizer Display System and Method

(76) Inventors: Rainer Wegenkittl, Sankt Poelten (AT); Donald K. Dennison, Waterloo (CA); John J. Potwarka, Waterloo (CA); Lukas Mroz, Wien (AT); Armin Kanitsar, Wien (AT); Gunter Zeilinger, Wien (AT)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 11/562,755

(22) Filed: Nov. 22, 2006

(51) Int. Cl. A61B 5/05; G01N 24/08

(52) U.S. Cl. 600/416; 324/312; 600/410

(57) ABSTRACT

A system and method for dynamically generating a localizer image comprising a plurality of pixel values. First, a plurality of planar images is provided, wherein each image has a plane. A three dimensional volume is generated based on the plurality of planar images, wherein the three dimensional volume comprises a plurality of values. An orientation and location are selected for the localizer image. Finally, a localizer image is generated, based on the plurality of values of the three dimensional volume, in the selected orientation and location.

### FIELD

The embodiments described herein relate to localizer systems and methods and more particularly to a system and method of dynamically generating and displaying localizer images

### BACKGROUND

Commercially available image viewing systems in the medical field utilize various techniques to present visual representations of image data to a user. Specifically, image data produced within modalities such as Computed Tomography (CT) and the like is displayed on a display terminal for review by a medical practitioner at a medical treatment site. In order for a medical practitioner to properly analyze image data in three dimensions, image data is typically presented in various multi-planar views each having a particular planar orientation.

FIG. 1A illustrates the conventionally known standard anatomical position that is utilized to provide uniformity to modality images. The standard anatomical position is where the subject is standing, feet together pointing forward, palms forward (no bones crossed), arms at sides, looking forward. As is conventionally, known, no matter what position a bone or skeleton is found in, surfaces are referred to as if the subject is standing erect in this standard anatomical position.

The various planes of reference are defined within this standard anatomical position, namely sagittal (FIG. 1B), coronal (FIG. 1C), transverse (or axial) (FIG. 1D) and oblique (FIG. 1E). As shown in FIG. 1B, the sagittal reference plane divides the subject into right and left halves. FIG. 1C illustrates the coronal plane or the frontal plane. As shown, the coronal reference plane divides the subject into anterior and posterior halves and is oriented at right angles to the sagittal reference plane. FIG. 1D illustrates the transverse reference plane or the axial reference plane. As shown, the transverse reference plane has a horizontal planar orientation and slices through the subject at any height. Generally speaking the transverse reference plane is perpendicular or orthogonal to the sagittal (FIG. 1B) and coronal (FIG. 1C) reference planes. It should be understood that in the case of an organ or other structure, a transverse reference plane is at right angles to the long axis of the organ or structure. Finally, FIG. 1E illustrates an oblique reference plane. As shown, the oblique reference plane may lie at any angle in respect of the subject.

By comparing various multi-planar views of an image series, a medical practitioner can better determine the presence or absence of a medical condition (e.g. disease, tissue damage etc.) Many attempts to optimize the display and presentation of multi-planar image data for viewing by a medical practitioner have been made.

When viewing the planar images on a viewing system a medical practitioner will often rely on one or more localizer images, which are also known as scout images, scanograms, pilot scans, and topograms, for orientation purposes. The localizer image is an image of a portion of the body that includes the area from which the image series is taken. The localizer image may be used by the medical practitioner to navigate through the image series. The localizer image may for example display a line, known as a scout line, to indicate where the plane of a currently viewed image intersects the localizer image. Given that this would only work well for planes that are not parallel, it is often the case that two localizer images are used. For example, one localizer image may be parallel to the sagittal plane and the other localizer image may be parallel to the coronal plane. Thus, regardless of the actual orientation of the plane of the medical image being viewed, it will not be parallel to at least one the localizer images and can therefore be displayed as a line on that localizer image.

Localizer images are generally generated at the same modality at which the image series are generated. For example, the localizer images may be created by performing low dose scans of an area larger than the area from which the image series is taken. It is sometimes the case the localizer images are sent to an image viewing system along with the image series. However, it is often the case that the localizer images are not sent to the image viewing system. In such a case, the medical practitioner may find it difficult to navigate through the images. In addition, even if localizer images are sent to the image viewing system, the medical practitioner may prefer to have the localizer images oriented in a different manner, depending on the orientation of the features of interest to the medical practitioner.

### SUMMARY

The embodiments described herein provide in one aspect, a method of dynamically generating a localizer image comprising a plurality of pixel values, the method comprising:

(a) providing a plurality of planar images, wherein each image is associated with a plane;

(b) generating a three dimensional volume based on the plurality of planar images, wherein the three dimensional volume comprises a plurality of values;

(c) selecting an orientation and location for the localizer image; and,

(d) generating a localizer image in the selected orientation and location based on the plurality of values of the three dimensional volume.

The embodiments described herein provide in another aspect, a system for dynamically generating a localizer image comprising a plurality of pixel values, the system comprising:

(a) a memory for storing a plurality of planar images, wherein each image has a plane;

(b) a processor coupled to the memory, said processor configured for:
(i) generating a three dimensional volume based on the plurality of planar images, wherein the three dimensional volume comprises a plurality of values;
(ii) selecting an orientation and location for the localizer image; and,
(ii) generating a localizer image in the selected orientation and location based on the plurality of values of the three dimensional volume.

Further aspects and advantages of the embodiments described herein will appear from the following description taken together with the accompanying drawings.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIGS. 1A, 1B, 1C, 1D, and 1E are schematic drawings illustrating the planar orientation of the sagittal, coronal, transverse (axial) and oblique reference planes within a human subject;

FIG. 2 is a block diagram of an exemplary embodiment of a localizer display system;

FIG. 3 is a schematic diagram of the localizer display system interface generated by the localizer display system of FIG. 2;

FIG. 4 is a flowchart diagram of an example set of operational steps executed by the localizer display system of FIG. 2;

FIG. 5 is a flowchart diagram of an example set of operational steps executed by the three dimensional volume generation module of FIG. 2;

FIG. 6 is a flowchart diagram of an example set of operational steps executed by the localizer image generation module of FIG. 2 when dynamically generating localizer images.

FIG. 7 is a flowchart diagram of an example set of operational steps executed by the localizer image generation module of FIG. 2 when generating planar localizer images; and,

FIG. 8 is a flowchart diagram of an example set of operational steps executed by the localizer image generation module of FIG. 2 when generating projection localizer images.

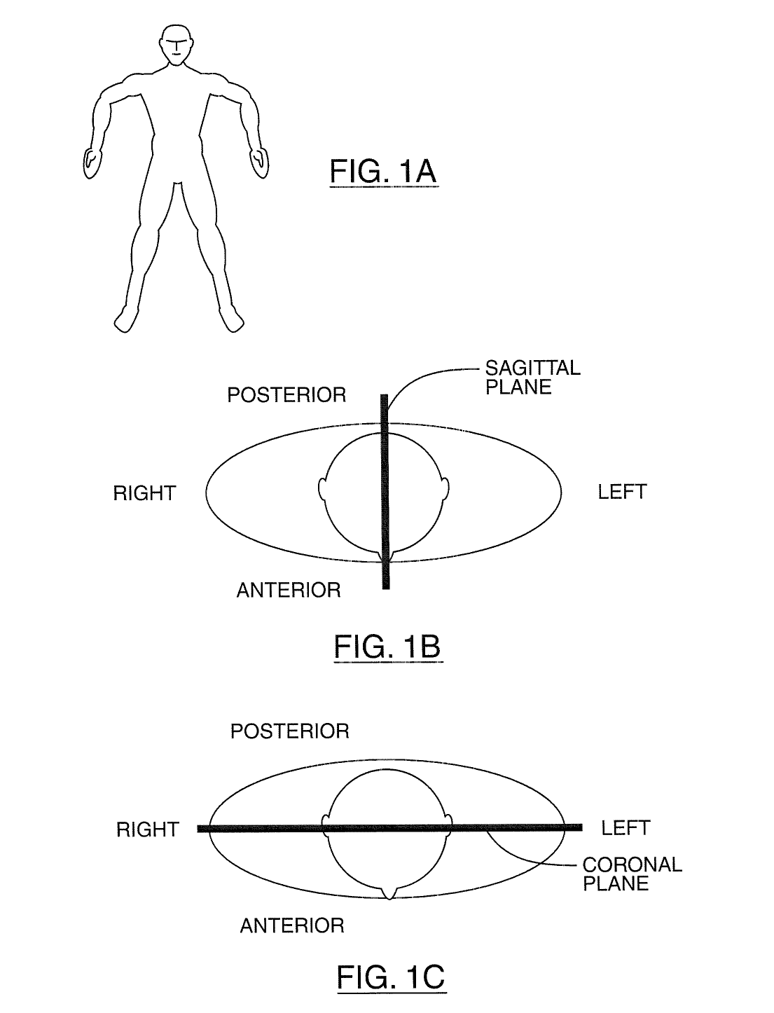

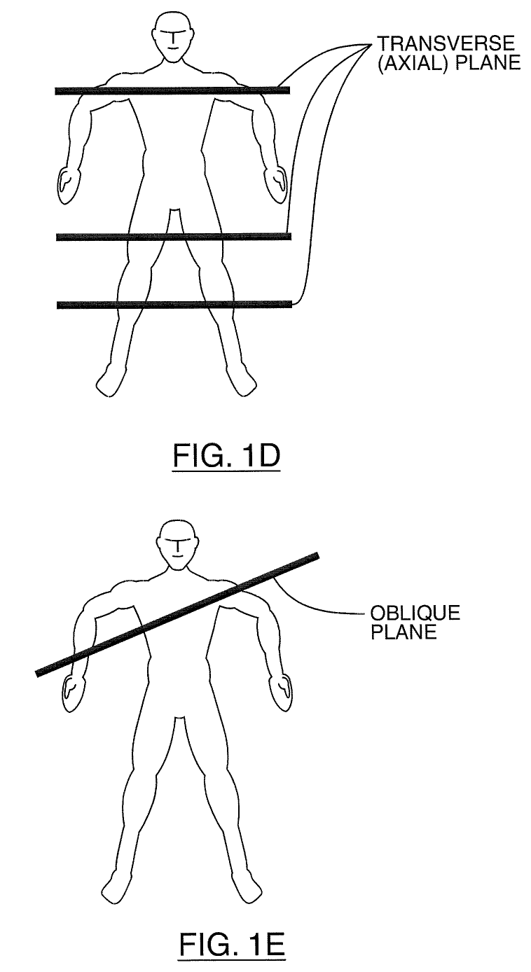

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION

It will be appreciated that for simplicity and clarity of illustration, where considered appropriate, numerous specific details are set forth in order to provide a thorough understanding of the exemplary embodiments described herein However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of the various embodiments described herein.

The embodiments of the systems and methods described herein may be implemented in hardware or software, or a combination of both. However, preferably, these embodiments are implemented in computer programs executing on programmable computers each comprising at least one processor, a data storage system (including volatile and non-volatile memory and/or storage elements), at least one input device, and at least one output device. For example and without limitation, the programmable computers may be a personal computer, laptop, personal data assistant, and cellular telephone. Program code is applied to input data to perform the functions described herein and generate output information. The output information is applied to one or more output devices, in known fashion.

Each program is preferably implemented in a high level procedural or object oriented programming and/or scripting language to communicate with a computer system. However, the programs can be implemented in assembly or machine language, if desired. In any case, the language may be a compiled or interpreted language. Each such computer program is preferably stored on a storage media or a device (e.g. ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like The computer useable instructions may also be in various forms, including compiled and non-compiled code.

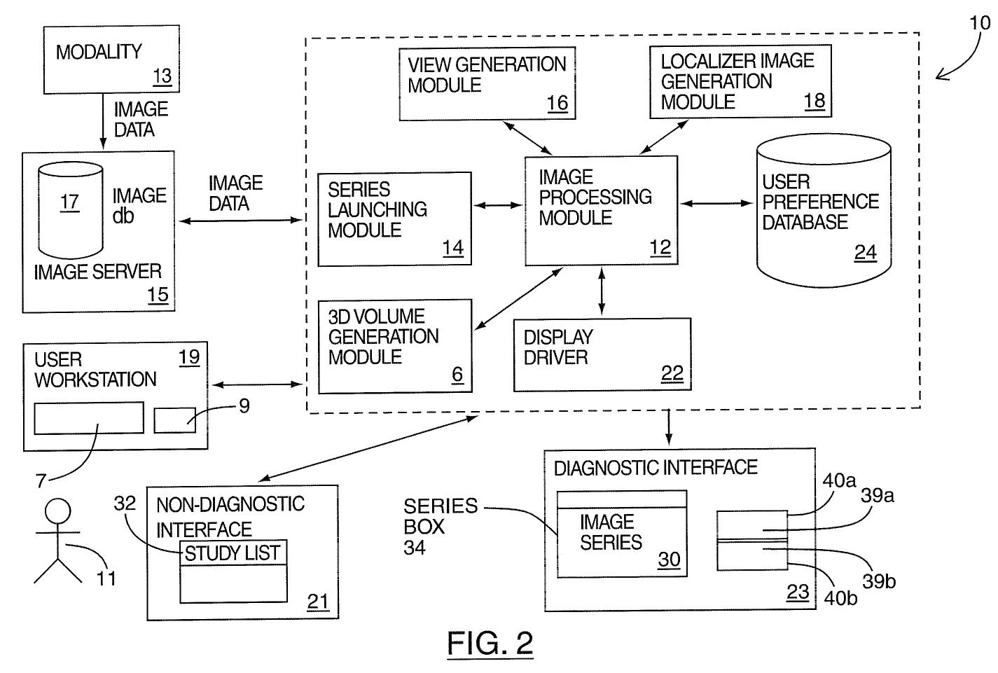

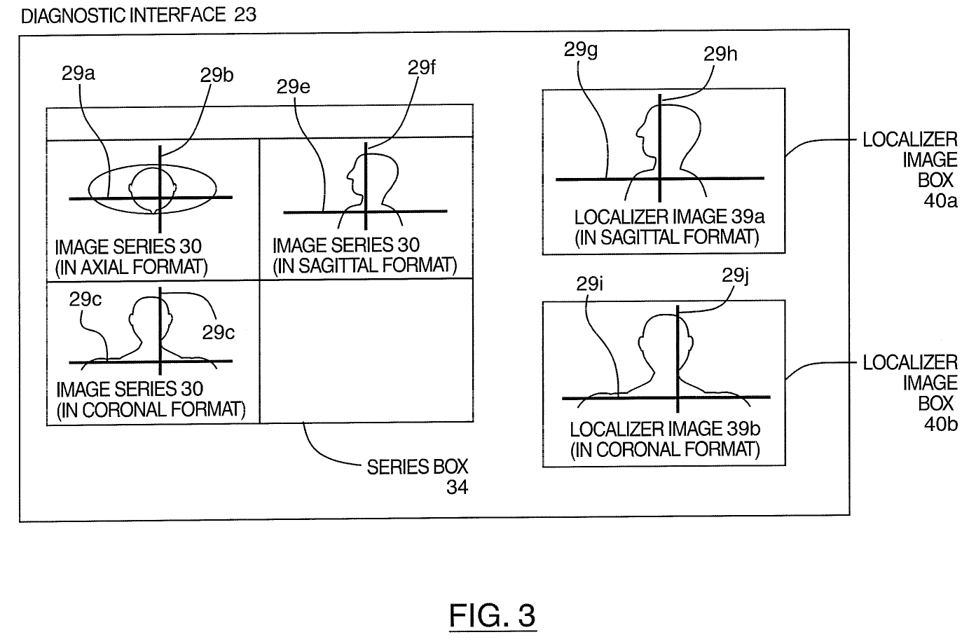

FIGS. 2 and 3 illustrate an exemplary embodiment of a localizer display system 10. Specifically, the localizer display system 10 includes an image processing module 12, a series launching module 14, a three dimensional volume generation module 6, a view generation module 16, a localizer image generation module 18 and a display driver 22.

As discussed in more detail above, it should be understood that localizer display system 10 may be implemented in hardware or software or a combination of both. Specifically, the modules of localizer display system 10 are preferably implemented in computer programs executing on programmable computers each comprising at least one processor, a data storage system and at least one input and at least one output device. Without limitation the programmable computers may be a mainframe computer, server, personal computer, laptop, personal data assistant or cellular telephone. In some embodiments, localizer display system 10 is implemented in software and installed on the hard drive of user workstation 19 and on image server 15, such that user workstation 19 interoperates with image server 15in a client-server configuration. In other embodiments, the localizer display system 10 can run from a single dedicated workstation that may be associated directly with a particular modality 13. In yet other embodiments, the localizer display system 10 can be configured to run remotely on the user workstation 19 while communication with the image server 15occurs via a wide area network (WAN), such as through the Internet.

As shown, image data associated with an image series 30 (i.e. a series of medical exam images) is generated by a modality 13 and stored in an image database 17 on an image server 15 for retrieval and display on diagnostic interface 23. User 11 selects or “launches” an image series 30 from study list 32 on non-diagnostic interface 21 in a selected initial planar orientation (e.g. sagittal, coronal, or axial orthogonal views, or a selected oblique view) using series launching module 14 and view generation module 16. The image series 30 selected for viewing by user 11 will be referred to as the “launching series”. Series launching module 14 retrieves image data that corresponds to the image series 30 selected for viewing and provides it to view generation module 16.

Three dimensional volume generation module 6 generates a three dimensional volume from the image data that is stored in image database 17, as will be explained below. View generation module 16 generates the image series 30, by utilizing the three dimensional volume, in an initial planar orientation (e.g. sagittal, coronal, or axial orthogonal views, or a selected oblique view) selected by the user 11 or through a default system as will be explained. Similarly, localizer image generation module 18 dynamically generates localizer images in an initial orientation and location from the three dimensional volume. The localizer images may be of various types including but not limited to projection localizer images and planar localizer images.

Examples of projection localizer images include maximum, mean and minimum intensity projection images. Examples of planar localizer images include multi-planar reformatting images, which are also known as multi-planar reformatted, (MPR) images. However, it should be understood that the localizer images 39 can be of other types and that these examples are meant to be illustrative only. The image characteristics such as the image type and image orientation and location depend on the initial settings selected by user 11 or through a default system as will be explained. Image processing module 12 then displays the launching series 30 in the initial planar orientation along with the dynamically generated localizer images 39.

User workstation 19 includes a keyboard 7 and a user pointing device 9 (e.g. mouse) as shown in FIG. 2. It should be understood that user workstation 19 may be implemented by any wired or wireless personal computing device with input and display means (e.g. conventional personal computer, laptop computing device, personal digital assistant (PDA), wireless communication device, etc.). User workstation 19 is operatively connected to non-diagnostic interface 21 and diagnostic interface 23. As described above, in one exemplary embodiment, localizer display system 10 is preferably installed on the hard drive of user workstation 19 and on image server 15, such that user workstation 19 interoperates with image server 15 in a client-server configuration.

Non-diagnostic interface 21 displays a study list 32 to user 11. Study list 32 provides a textual format listing of image series 30 that are available for display. Study list 32 also includes associated identifying indicia (e.g. body part, modality, etc.) and organizes image series 30 in current and prior study categories Other associated textual information (e.g. patient information, image resolution quality, date of image capture, etc.) is simultaneously displayed within study list 32 to further assist the user 11 in selection of image series 30. Typically, user 11 will review study list 32 and select a desired listed image series 30 for display on diagnostic interface 23. Non-diagnostic interface 21 is preferably provided on a conventional color computer monitor (e.g. a color monitor with a resolution of 1024×768) with sufficient processing power to run a conventional operating system (e.g. Windows NT). High resolution graphics are not typically necessary for non-diagnostic interface 21 since this display is usually only displaying textual information to user 11.

Diagnostic interface 23 provides high resolution image display of image series 30 and localizer images 39 to user 11. Image series 30 is displayed within a series box 34. Diagnostic interface 23 is preferably provided on medical imaging quality display monitors with relatively high resolution typically used for viewing CT image studies (e.g. black and white “reading” monitors with a resolution of 1280-1024 and up).

Display driver 22 is a conventional display screen driver implemented using commercially available hardware and software. As shown in FIG. 2, display driver 22 ensures that image series 30 and localizer images 39 are displayed in a proper format on diagnostic interface 23. Specifically, image series 30 are displayed within series boxes 34 and localizer images 39 are displayed within localizer boxes 40a and 40b Each series box 34 contains an image series 30 and each localizer box 40 contains a localizer image 39. Display driver 22 provides image data associated with image series 30 and localizer images 39 appropriately formatted so that image series 30 are properly displayed within one or more series boxes 34 and localizer images 39 are properly displayed within localizer boxes 40 on diagnostic interface 23.

Modality 13 is any conventional image data generating device (e.g. computed tomography (CT) scanners, etc.) utilized to generate image data that corresponds to patient medical exams. A medical practitioner utilizes the image data generated by modality 13 to make a medical diagnosis (e.g. for investigating the presence or absence of a diseased part or an injury or for ascertaining the characteristics of the diseased part or the injury). Modalities 13 may be positioned in a single location or facility, such as a medical facility, or may be remote from one another. Image data from modality 13 is stored within image database 17 within an image server 15 as conventionally known.

Image processing module 12 coordinates the activities of series launching module 14, three dimensional volume generation module 6, view generation module 16 and localizer image generation module 18 in response to commands sent by user 11 from user workstation 19 and stored user display preferences from user display preference database 25. When user 11 launches an image series 30 from study list 32 on non-diagnostic interface 21, user 11 may select an initial planar orientation for the image series 30 and an initial type and orientation and location for localizer image 39.

Once the series is launched, image processing module 12 instructs series launching module 14 to retrieve image data that corresponds to the selected image series (i.e. the “launching series”) and to provide it to three dimensional volume generation module 6. Three dimensional volume generation module 6 generates a three dimensional volume from the image data and provides it to view generation module 16. Image processing module 12 then instructs view generation module 16 to generate planar images from the three dimensional volume. Since user 11 has also selected an initial planar orientation (e.g. sagittal, coronal, or axial orthogonal views, or oblique view), view generation module 16 generates the image series 30 in the initial planar orientation.

Finally, image processing module 12 then instructs localizer image generation module 18 to dynamically generate localizer images 39. Similar to the above, since user 11 has also selected an initial type and orientation and location for the localizer images 39, localizer image generation module 18 generates the localizer images 39 to be of an initial type (such as projection or planar) and in the initial orientation and location.

Series launching module 14 is utilized by image processing module 12 to retrieve image data from image server 15 associated with the selected image series 30 for display on diagnostic interface 23. The particular initial planar orientation for the launching series 30 is determined on the basis of user preference (explicit or preferred) or on the basis of default system preferences. When user 11 launches an image series 30 from study list 32 on non-diagnostic interface 21, image processing module 12 instructs series launching module 14 to retrieve image data that corresponds to the launching series 30 and to provide it to view generation module 16.

Series launching module 14 allows user 11 to explicitly request a particular initial planar orientation (e.g. axial, coronal, sagittal or oblique) for an image series 30 from study list 32. Similarly, series launching module 14 allows user 11 to explicitly request particular initial settings for the localizer images 39, such as the number, type and orientation and location of the images. User 11 may also establish a default initial planar orientation preference for image series 30 and default initial orientation and location and type preferences of localizer images 39 in the user preference database 24. Such viewing format preferences would be utilized in the case where no explicit selection of an initial planar orientation or localizer orientation and location and type is made by user 11. For example, user 11 may establish a default initial planar orientation preference within user preference database 24 for all image series 30 to be initially displayed in a coronal initial planar orientation. In such a case, any image series 30 launched without an explicit initial planar orientation selection will be launched on diagnostic interface 23 in a coronal planar view format. Similarly, user 11 may establish the default initial number of localizer images 39 to be two, that they are both planar images and that they are oriented and located in the axial and sagittal planes.

Series launching module 14 also provides for the ability to establish system-wide or multi-user (i.e. departmental) initial default settings. These kinds of initial default settings would preferably be applied when no explicit initial planar orientation for image series 30 and no explicit initial settings for localizer images 39 are selected on launch and when no user default has been established. For example, a departmental viewing format default could be established by a CT specialty department in a hospital such that on start-up and in the absence of any user defaults or explicit user selections, an image series 30 is launched in coronal initial planar orientation and that two planar localizer images 39 be launched in the axial and sagittal planes.

Also, it should be understood that it is contemplated that series launching module 14 would monitor the initial default settings and viewing format selections a user 11 or a group of users 11 makes in previous imaging sessions and store related preferences in preference database 24. This includes the viewing settings for image series 30 and the settings for the localizer images 39. Accordingly, when an image series is launched, viewing format preferences for the series 30 as well as for the localizer images 39 established in a previous session would be utilized.

View generation module 16 also generates scout lines 29 (FIG. 3) within localizer images 39. In some embodiments, view generation module 16 can optionally generate scout lines 29 within image series 30 as well as localizer images 39. Scout lines 29 can be used to indicate the location and progress of image series 30 in one planar orientation in reference to image series 30 in another planar orientation and also in reference to localizer images 39. In some embodiments, separate localizer images 39 are not generated and instead each view of image series 30 serves as a localizer image. This is possible because each view of image series 30 can display scout lines for each of the other images.

Reference is now made to FIG. 3, which is a schematic diagram of various embodiments of the display of localizer display system 10. Illustrated therein is diagnostic interface 23, with series box 34 and a first localizer image box 40a and a second localizer image box 40b. Series box 34 is shown as displaying three images of image series 30 each of which is in a different format. Specifically, the axial, sagittal and coronal formats are shown. Furthermore, each localizer image box 40a and 40b is shown as displaying localizer image 39a and 39b respectively.

Although FIG. 3 illustrates the localizer images 39a and 39b as being in separate boxes from the series box 34, in some embodiments image series 30 and localizer images 39a and 39b could be displayed in the same box. Furthermore, it is not necessary that both localizer images be displayed at the same time. For example, in some embodiments a single localizer image could be displayed in the empty bottom right corner of series box 34, and although only one localizer image is displayed at a time user 11 can cycle between the localizer images 39a and 38b by inputting appropriate commands. In addition, it should be understood that in various embodiments more or less images from image series 30 can be displayed in series box 34 in various planar orientations. In addition, in various embodiments all localizer images and images from image series 30 may be displayed in the same box, such as series box 34.

Also illustrated in FIG. 3 are a variety of scout lines, 29a to 29j. Each scout line 29 is used to indicate the location and progress of image series 30 in one planar orientation in reference to image series 30 in other planar orientations and the localizer images 39. Each scout line 29 can be said to have an associated planar image. The associated image is the image for which scout line 29 is representative. For example, scout lines 29a, 29f and 29h are representative of image series 30 and therefore, the image of image series 30 in coronal format is the associated image. Specifically, scout lines 29a, 29f and 29h indicate the line of intersection between the plane of the associated image and the plane of the image on which they appear. Thus, scout line 29a indicates the line of intersection between the plane of the currently displayed image series 30 in the coronal format and the plane of currently displayed image series 30 in the axial format. Similarly, scout lines 29b, 29d, and 29j are associated with image series 30 in the sagittal format. Lastly, scout lines 29c, 29e and 29i are associated with image series 30 in the axial format.

As mentioned above, scout lines 29 are used to indicate the line that is formed by the intersection of the plane of the associated image with the plane of the image on which the scout line is displayed. This is possible because two non-parallel planes intersect in a line. Thus, as the view of a particular image of image series 30 is altered the associated plane may be different and therefore the line of intersection between that image and the other displayed images of image series 30 will also be different. This in turn means that the scout line 29 will have to be redrawn on the other displayed views of image series 30.

Thus, as the view of any of the displayed images of image series 30 is altered, view generation module 16 updates that image's corresponding scout line 29 in each of the other views of image series 30 as well as in each localizer image 39. Thus, if one of the views of image series 30 is altered such that the new view displays a plane that is adjacent to the plane in the old view, then the new scout line 29 corresponding to this view of image series 30 will be shifted with respect to the old scout line 29.

In various embodiments, localizer images 39 do not have corresponding scout lines 29. Localizer images 39 are generally only used as navigational tools to assist user 11 in orienting himself or herself while viewing image series 30. Furthermore, localizer images 39 are generally not altered much. Therefore, generally user 11 would not require scout lines 29 for the localizer images 39.

In addition, in various embodiments, localizer images 39 may be non-planar images. For example, projection localizer images 39 are non-planar images. Such images can display features that exist in a multiple planes. Therefore, given that scout lines 29 are used to illustrate the intersection of two planes, it does not make sense to have an associated scout line 29. The reason for this is that the features illustrated in a projection image do not all exist in a particular plane and therefore it is not possible to draw a line of intersection in the way that it is possible for planar images.

The fact that a projection localizer image 39 does not occupy a particular plane presents another challenge. Specifically, as was explained above, the features illustrated in a projection localizer image 39 do not exist in a single plane. In fact it can be said that a projection localizer image 39 illustrates features that exist in a plurality of parallel planes. Furthermore, each pixel of a projection image can be said to exist in one of the plurality of parallel planes. Each of these parallel planes can be said to be parallel to the surface (e.g. flat panel display) on which the projection image is displayed. Equivalently, it could be said that each of these parallel planes is perpendicular to the direction of the image. The direction of the image can be defined as the direction in which a hypothetical camera lens would point in order to capture the features of the image.

Given the above, it is generally not possible to define a single line of intersection between the projection image 39 and another plane. The reason is that there would generally be a plurality of intersecting lines, one line for each plane, rather than a single intersecting line. However, for planes that are perpendicular to the parallel planes mentioned above, it is possible to draw a scout line 29. The reason for this follows from the fact that such a perpendicular plane would intersect each plane of projection localizer image 39, in a parallel line. Each of these parallel lines would appear as superimposed over each other. Therefore, it is possible to draw a single scout line 29 in the projection localizer image 39 that is representative of each of the intersecting lines.

In some embodiments, the difficulties associated with projection localizer images 39 not being planar are dealt with by selecting a representative plane. The representative plane could for example be a plane that is orthogonal to the direction of the localizer image 39 and is approximately midway between the plane of the closest pixel and the plane of the furthest pixel that appear in the localizer image 39. For example, if the localizer image illustrates a frontal view of the head of a patient, then the representative plane could be a plane, such as the coronal plane, which cuts through the middle of the head.

In various embodiments, three dimensional volume generation module 6, generates a three dimensional volume from image data that is stored in image database 17 on image server 15. The image data that is utilized is generally a series of planar medical images. In some embodiments, three dimensional volume generation module 6 generates the three dimensional volume from images that are aligned along an axis that is normal to the plane of the images. If any of the images are not aligned then they are discarded and not used by three dimensional volume generation module 6 in creating the three dimensional volume. Each image represents a plane that cuts through the three dimensional volume.

Furthermore, each aligned image represents a parallel plane in the three dimensional volume. In other embodiments, three dimensional volume generation module 6 is capable of creating a three dimensional volume from unaligned images. Thus, as will be explained in greater detail below, three dimensional volume generation module 6 analyzes each image and generates a three dimensional volume based on the images. The three dimensional volume that is generated utilizes voxels to represent values in a three dimensional grid. As used herein, the term voxel refers to a volume element that is applicable for higher order interpolation such as tri-linear interpolation. For example, a three dimensional volume defined by a regular grid where the grid defines cells and where each corner of a cell holds a value would be acceptable.

Localizer image generation module 18 is used to generate localizer images 39 from the three dimensional volume created by three dimensional volume generation module 6. As will be explained in greater detail below, localizer image generation module 18 allows for the creation of different types of localizer images 39 in any orientation with respect to the three dimensional volume In various embodiments, localizer image generation module 18 can create either planar localizer images 39 or projection localizer images 39.

Planar localizer images 39 are similar to the images in image series 30, in that they are simply planar images that appear as a cross section of the medical subject. Projection localizer images 39 are images that are not cross sections and therefore display features that are not necessarily coplanar. Thus, in some embodiments, projection localizer images 39 may appear similar to x-ray images or other comparable images. In the case of planar localizer images 39, image generation module 18 can create the planar images in any plane of the three dimensional volume. As will be explained below, this can be done through the use of any appropriate technique, such as interpolating values in the three dimensional volume to generate pixel values in the plane of the planar localizer image 39.

In the case of projection localizer images 39, image generation module 18 can create projection localizer images 39 in any orientation and location with respect to the three dimensional volume. As will be explained below, this can be done by through the use of any appropriate technique such as ray casting, creating a “thick MPR” image, or volume rendering. It is important to note that the use of the term ray casting is not intended to imply that projection localizer images 39, are perspective projection images. In particular, in some embodiments projection localizer images 39 are parallel projection images. The term “thick MPR” refers to a thick slab that is cut through the volume and can be generated by any appropriate technique, such as by combining the voxels that are perpendicular to the slab by, for example, taking their mean or maximum value.

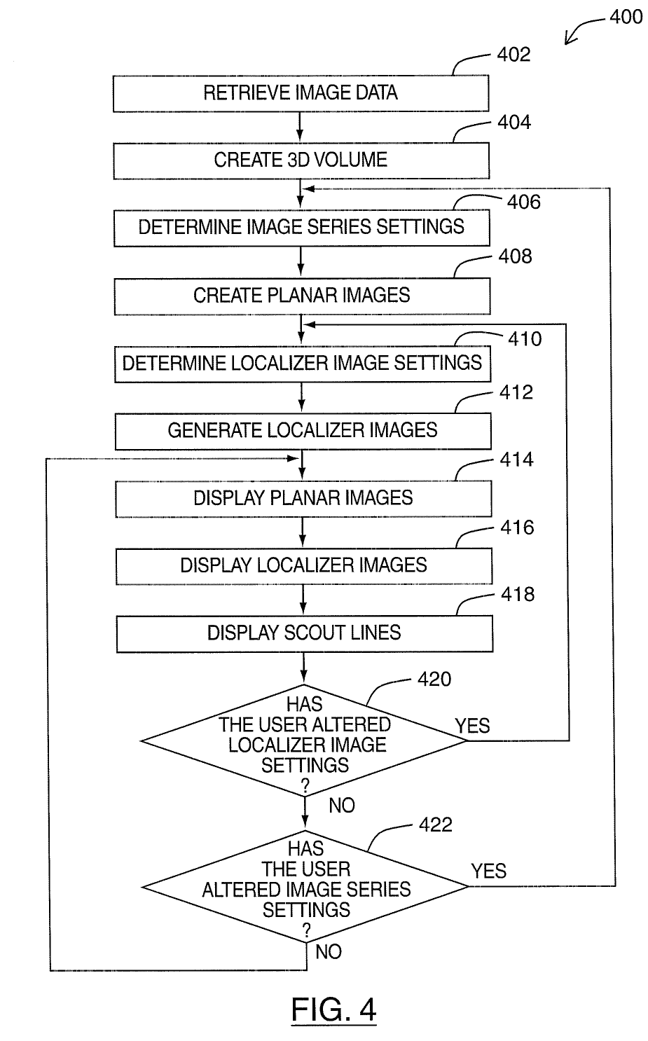

Reference is now made to FIG. 4, which is a flowchart diagram that illustrates the basic operational steps 400 taken by the localizer display system 10 when generating and displaying localizer images 39.

At step (402), series launching module 14 is utilized by image processing module 12 to retrieve the image data from image server 15. As mentioned above, the image data may be received from any appropriate source such as, for example but not limited to, modality 13. The image data that is retrieved depends on the instructions inputted by user 11. Specifically, the image data associated with the image series 30, which was selected by user 11 from the study list 32 on non-diagnostic interface 21. After step (402) is completed, step (404) is executed.

At step (404), image processing module 12 instructs series launching module 14 to provide three dimensional volume generation module 6 with the image data retrieved in the previous step. Also at this step, three dimensional volume generation module 6 utilizes the image data to construct a three dimensional volume. After step (404) is completed, step (406) is executed.

At step (406), the settings for image series 30 such as the initial planar orientation for image series 30 is determined. As was mentioned above, these settings may be imputed by user 11 or they may be retrieved from the user preference database 24.

At step (408), the planar images are created from the three dimensional volume. This may be accomplished by any appropriate method such as tri-linear interpolation of the 8 closest voxels.

At step (410), the localizer image settings are determined. In some embodiments this may include, for example but is not limited to, the number of localizer images 39, the type of localizer images, and the orientation and location of each localizer image 39. As with the initial settings for the image series 30, the initial localizer image settings may be requested by user 11 or they may be retrieved from the user preference database 24.

Then at step (412), localizer image generation module 18 generates each localizer image 39. This may be accomplished by any appropriate method depending on the type of localizer image 39 that is to be generated, as will be discussed in greater detail below.

At step (414), image processing module 12 instructs display driver 22 to cause the planar images of image series 30 to be displayed on diagnostic interface 23.

At step (416), image processing module 12 instructs display driver 22 to display the localizer images 39 on diagnostic interface 23.

At step (418), image processing module 12 causes scout lines 29 to be displayed over the localizer images 39 and planar images. As explained above, the scout lines 29 are used to represent the planes of the various images of image series 30 that are displayed on diagnostic interface 23. Scout lines 29 can be displayed on localizer images 39 as well as on each image of image series 30 that is displayed on diagnostic interface 23. Thus, each image displayed on diagnostic interface 23, may have several scout lines 29 displayed on it, each of which represents each of the other non-parallel currently displayed views of image series 30 that is displayed on diagnostic interface 23. As any of the displayed views of image series 30 is altered (i.e. a different orientation or plane is selected), view generation module 16 causes the corresponding scout line 29 in each of the other images to be updated.

At step (420), it is determined whether the user has a decided to alter the localizer image settings. Given that localizer display system 10 is able to dynamically generate localizer images 39, the user 11 is given the ability to alter the settings for the localizer images 39 and thereby cause the system to generate new localizer settings. The user may wish to alter the localizer image settings for a number of reasons. For example, the orientation and location of the present localizer images 39 may not be appropriate given the orientation of the area of interest to the medical professional. For example, a medical practitioner may like to generate and view planar images of image series 30 in the sagittal plane. The current localizer image 39 may be in the sagittal plane as well. Given that the localizer image 39 and the displayed image of image series 39 are in parallel planes, the localizer image 39 will not be of much use to user 11, as no scout lines will be able to be displayed on the localizer image 39 in order to help user 11 navigate through the image series 30. Since the scout line 29 represents the line at which two planes intersect, it would not make much sense to draw a scout line 29 for two planes that are parallel. The localizer image 39 is of most use when its orientation is at an angle with respect to the plane of the image series 30.

Under such conditions a scout line 29 representing the plane may be displayed on the localizer image 39. Thus, in such a case user 11 may prefer to have at least one of two localizer images 39, either one parallel to the coronal plane or one parallel to the axial plane or both, as this would provide him or her with the best ability to orient himself or herself while viewing the image series 30. Thus, user 11 may wish to regenerate at least one localizer image 39 according to the above-described settings.

If user 11 has not chosen to alter the localizer image settings, then step (422) is executed. If user 11 has chosen to alter the localizer image settings, then step (410) is repeated based on the choices made by user 11.

At step (422), it is determined whether the user has altered the image series settings. If not, then step (414) is repeated. If yes, then step (422) is repeated based on the choices made by the user.

It is important to note that FIG. 4 is intended to be exemplary only and the steps illustrated do not have to be executed in the order shown. In particular, in various embodiments, many of the steps may be executed in a different order or in parallel with each other. For example, in some embodiments, the planar images are displayed while the localizer images are being generated or if the planar images are already available, then they may be displayed while the three dimensional volume is being generated. This may be done in order to minimize delays associated with the generation of the three dimensional volume and the localizer images 39 by allowing user 11 to view and work with the planar images as soon as possible. Once the localizer images are ready they are displayed along with the appropriate scout lines.

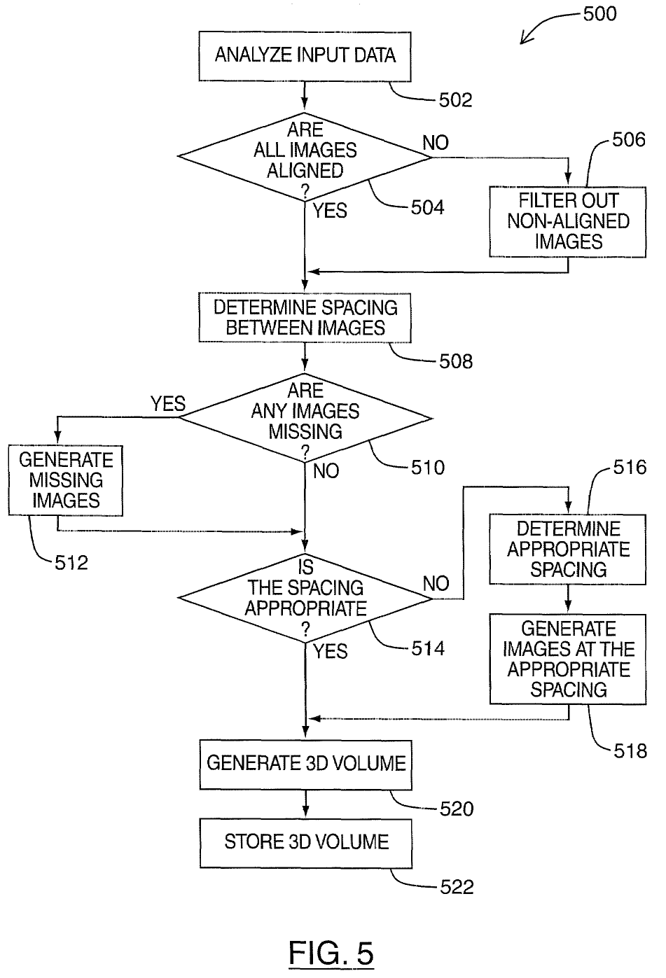

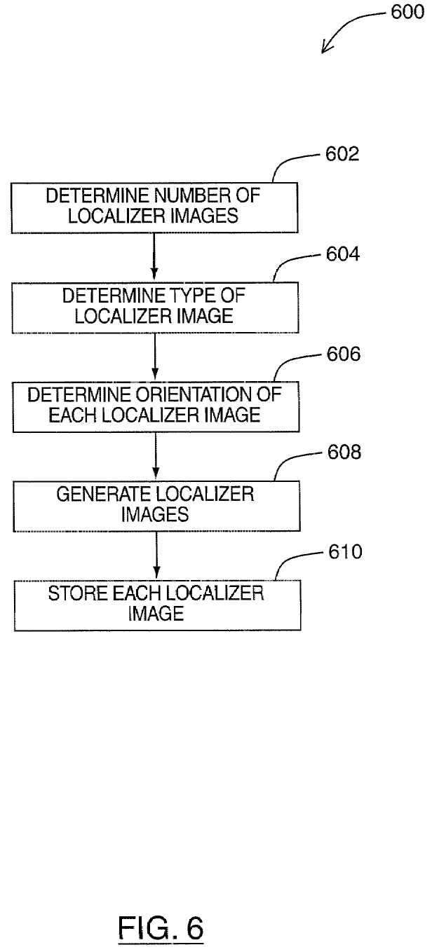

Reference is now made to FIG. 5, which is a flowchart diagram that illustrates the basic operational steps 500 taken by the three dimensional volume generation module 18 when generating a three dimensional volume.

At step (502), the image data is analyzed by the three dimensional volume generation module 6. At step (504) three dimensional volume generation module 6 determines whether all the image data is aligned. Generally, the image data that is generated by modality 13 and stored on image server 15 comprises a series of planar images that are aligned in some manner. This does not imply that each of the images is necessarily aligned in such a manner that the edges of each image are aligned along an axis that is normal to the plane of each of the images. In particular, each image may be offset with respect to adjacent images by a given amount.

Generally, the offset between successive images is substantially constant, assuming a constant spacing between images. This could for example represent a situation in which the scan performed by modality 13 is skewed with respect to the body. In such a situation, each image may still be referred to as being aligned even though the edges of successive images are offset with respect to each other since the offset is substantially constant between consecutive images. Thus, generally each image in the image data represents a two dimensional slice of the three dimensional volume that is to be created. If three dimensional volume generation module 6 determines that each image is not properly aligned, then step (506) is executed. If on the other hand, three dimensional volume generation module 6 determines that each image is aligned, then step (508) is executed.

At step (506), three dimensional volume generation module 6 discards the images that are not properly aligned with respect to the other images. The discarded images are not utilized by three dimensional volume generation module 6 for generating the three dimensional volume. After step (506) is completed, step (508) is executed. In various other embodiments, the image data need not be aligned and therefore, steps (504) and (506) are not preformed.

At step (508), three dimensional volume generation module 6 further analyzes the image data in order to determine the “spacing” between images. As mentioned above, generally, each image in the image data is generated by a modality by taking successive scans that are spaced along an axis. Thus, each image in the image data may be thought of as representing a “two dimensional slice” of the subject or as a portion of a plane that cuts through the subject. Thus, at this step three dimensional volume generation module 6 determines the spacing of the planes of each image in the image data, which is equivalent to determining the spacing between the planes of the images within the body of the patient.

At step (510), three dimensional volume generation module 6 determines whether or not any images are missing from the set of image data. The images may be missing for a number of reasons including but not limited to the possibility of having been discarded at step (506). This can be accomplished in any appropriate manner. For example, assuming a constant spacing between images, the images are analyzed in order to determine the spacing between each image and if the spacing between any two images is determined to be a multiple of the spacing between other consecutive images, then it is known that there is at least one image missing between those two images.

Furthermore, medical images produced by modalities often comprise information with respect to such things as pixel spacing and the image location within the patient coordinate space. This information is often stored according to the Digital Imaging and Communications in Medicine (DICOM) standard file format. This information may be used to determine the spacing between images.

If three dimensional volume generation module 6 determines that there are images that are missing, then step (512) is executed. If, on the other hand, three dimensional volume generation module 6 determines that no images are missing then step (514) is executed. At step (512), any missing slices are generated through interpolation. Any appropriate method of interpolation may be utilized. After step (512) is completed, step (514) is executed.

At step (514), three dimensional volume generation module 6 determines whether the spacing between images is appropriate. In some embodiments this step comprises three dimensional volume generation module 6 determining whether the images in the image data are evenly spaced. However, it should be understood that in various other embodiments the images in the image data need not be evenly spaced.

If three dimensional volume generation module 6 determines that the images are not appropriately spaced, then step (516) is executed. If three dimensional volume generation module 6 determines that the images are appropriately spaced, then step (520) is executed.

At step (516), three dimensional volume generation module 6 determines an appropriate spacing for the images in the image data. As mentioned above, some embodiments utilize image data that has an even spacing of images for the generation of the three dimensional volume. For such embodiments, step (516) comprises determining an appropriate even spacing given the current spacing of the images in the image data.

At step (518), three dimensional volume generation module 6 generates planar images for use with the image data at the above-determined appropriate spacing. Any appropriate method, including but not limited to, interpolation, may be used for the generation of the planar images.

At step (520), three dimensional volume generation module 6 generates the three dimensional volume from image data. Any appropriate method of generating the three dimensional volume may be utilized. For example, some embodiments utilize known methods such as those used by medical imaging systems when generating three dimensional volumes for the purpose of generating MPR images.

At step (522) the three dimensional volume is stored. In various embodiments the three dimensional volume need not be stored in a database. In such embodiments the three dimensional volume may be generated each time it is needed and then discarded. In some embodiments, the three dimensional volume will be temporarily cached and used as needed.

Reference is now made to FIG. 6, which is a flowchart diagram that illustrates the basic operational steps 600 taken by the localizer image generation module 18 when dynamically generating the localizer images 39.

At step (602), the number of localizer images 39 is determined. Localizer image generation module 18 allows user 11 to select the number of localizer images 39 that are utilized. Alternatively, the system may have its own default settings or default preferences that may have been set by user 11, which may be retrieved from user preference database 24. In various embodiments, the default number of localizer images 39 is two. However, in various other embodiments, the default number of localizer images 39 may be less or greater than two.

At step (604), the type of localizer image is selected. In various embodiments, user 11 may choose between a planar localizer image 39 and a projection localizer image 39. In some embodiments, the planar localizer image 39 is similar to the planar images generated for the displaying a particular planar orientation of image series 30. More specifically, in some embodiments both the displayed image series 30 and the localizer images 39 may be MPR images. In such embodiments, both these types of images may be generated in the same manner, as will be explained below. The projection localizer images 39 are images that display features that exist in more than one plane and resemble the localizer images 39 that are generated at some modalities.

At step (606), the orientation and location of each localizer image 39 is determined. Localizer image generation module 18 allows user 11 to select the orientation and location of each localizer image 39. Alternatively, if the user 11 does not make an explicit selection, default orientation and location settings may be retrieved from user preference database 24. If the localizer image 39 is a planar localizer image 39, then this step comprises selecting a plane in the three dimensional volume for the localizer planar image 39. More specifically, the orientation of the plane and the location (or position) of the plane with respect to the three dimensional volume is selected. On the other hand, if the localizer image 39 is a projection image, then this step comprises selecting a direction or orientation and location from which the three dimensional volume is to be viewed.

At step (608), localizer image generation module 18 generates each localizer image 39 from the three dimensional volume. The manner in which this is accomplished depends on the type of localizer image 39 that is desired. As explained above, in various embodiments, the localizer images 39 may be either planar localizer images 39 or projection localizer images 39. More specifically, as mentioned above, in some embodiments, the planar localizer image 39 is similar to the planar images of image series 30. Therefore, in such embodiments, planar localizer images 39 can be generated in the same manner as the planar series images 30. In some embodiments, this is accomplished through multi-planar reformatting, which is also known as multi-planar recasting. In various embodiments, this is accomplished by selecting a plane in the three dimensional volume and then for each pixel in the plane of the image, interpolating the eight nearest voxels of the three dimensional volume in order to obtain a pixel value.

In contrast, as mentioned above, the projection localizer images 39 can be generated by the use of techniques such as ray casting, creating a “thick MPR” image, or volume rendering. For example, a mean intensity projection image may be created by performing ray casting on of the three dimensional volume and the resulting image may be used to generate a projection localizer image 39. The ray casting is performed along the direction of the image. The direction of the image is determined by the orientation and location that was selected at step (606). Specifically, the direction of the image can be said to be in the same direction as a line that is normal to the screen on which the image is displayed or the direction in which a hypothetical camera lens would be placed in order to capture the features in the image as they appear.

At step (610), each localizer image 39 is stored and the process ends.

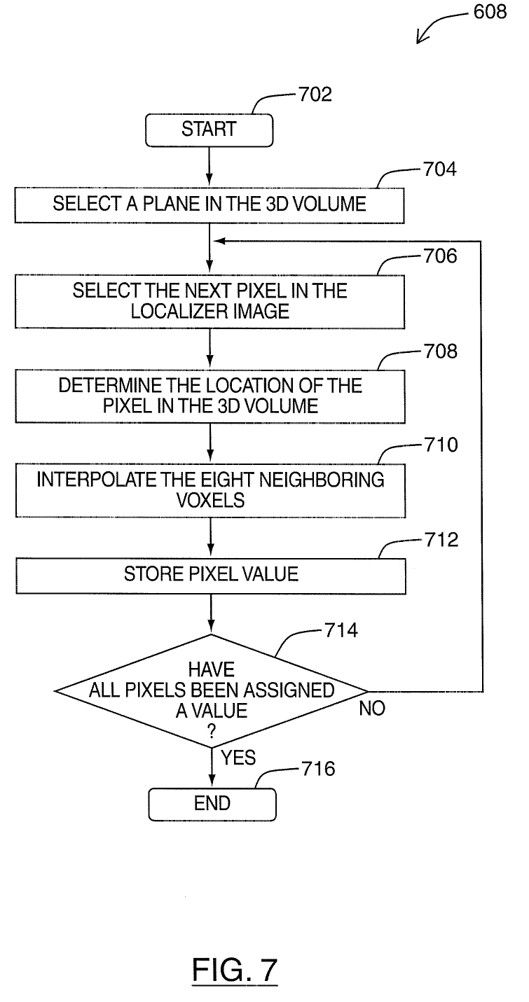

Reference is now made to FIG. 7, which is a flowchart diagram that illustrates the steps 608 taken by the localizer image generation module 18 when generating planar localizer images 39.

The process starts with step (702). At step (704), localizer image generation module 18 selects a plane that passes through the three dimensional volume. Both the location and orientation of the plane is selected. This plane will be used as the plane of the planar localizer image 39.

At step (706), a pixel in the localizer image 39 is selected. The pixel may be selected in any appropriate manner. For example, in some embodiments, the first pixel may be selected from one of the corners of the image and each adjacent pixel may be selected consecutively until the last pixel is reached.

At step (708), localizer image generation module 18 determines the location in the three dimensional volume corresponding to the pixel that was selected at step (704). This may be accomplished by any appropriate method.

At step (710), localizer image generation module 18 determines an appropriate value for the selected pixel. This step may be accomplished by any appropriate method. For example, if the three dimensional volume is represented by voxels then each pixel value may be determined through tri-linear interpolation of the 8 nearest voxels.

At step (712), the pixel value generated at step (710) is stored.

At step (714), localizer image generation module 18 determines whether or not all pixels in the localizer image 39 have been assigned a value. If yes, then the process ends at step (716). If not, then step (706) is repeated.

It should be understood that FIG. 7 is exemplary only and that other methods may be used to generate planar localizer images. In particular, in some embodiments, the relationship between a single pixel displacement in the localizer image and the corresponding displacement in the three dimensional volume is determined. This relationship is determined for two orthogonal vectors that define the plane of the image (e.g. x and y axes in the plane of the image). This provides an offset that can be multiplied by any pixel position in the image and thereby yield a corresponding position in the three dimensional volume. Thus, this may provide an efficient way of determining a pixel position in the three dimensional volume.

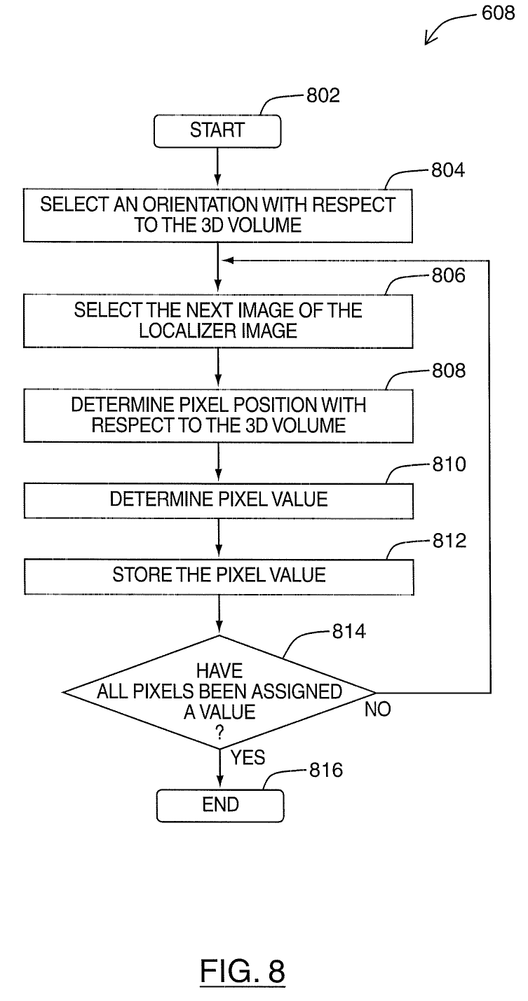

Reference is now made to FIG. 8, which is a flowchart diagram that illustrates the operational steps 608 taken by the localizer image generation module 18 when generating a projection localizer image 39.

The process begins at step (802). At step (804), localizer image generation module 18 selects an orientation and location for the projection localizer image 39 with respect to the three dimensional volume. For some embodiments, the orientation and location represents the perspective from which localizer image 39 illustrates the three dimensional volume. This step can be thought of as placing a notional lens somewhere outside of the three dimensional volume. The location represents the location of the hypothetical camera lens and the orientation represents the direction in which the camera lens is pointed. In contrast to planar localizer images 39 which illustrate a plane within the three dimensional volume, projection localizer images 39 illustrate the three dimensional volume from outside of the three dimensional volume. Thus, the selection of the orientation and location determine from which angle and location the three dimensional volume is illustrated.

The projection image can be of various types. For example, in some embodiments, the projection images 39 can be a perspective projection images; while, in various other embodiments projection localizer images 39 can be parallel projection images. Perspective projection images display features that are further away from the notional camera lens as being smaller than the features that are closer to the notional camera lens. In contrast, parallel projection images display features as being the same size regardless of their distance from the notional camera lens. In various embodiments, the type of the projection image can be selected by user 11 or can be determined according to settings in user preference database 24.

At step (806), a pixel in the localizer image 39 is selected. The pixel may be selected in any appropriate manner. For example, in some embodiments, the first pixel may be selected from one of the corners of the image and each adjacent pixel may be selected consecutively until the last pixel is reached.

At step (808), the pixel position, of the pixel selected at step (806), is determined with respect to the three dimensional volume. This may be accomplished by any appropriate method. For example, this step corresponds to determining the position of pixel on the notional camera lens mentioned in respect to step (804).

At step (810), the pixel value is determined. This can be accomplished by any appropriate method. For example, ray casting can be used to determine the pixel value. Ray casting involves projecting a ray from the position of the pixel, which was selected in the previous step, towards and through the three dimensional volume. The direction of the ray is dependent on the orientation of the image. Specifically, the ray is cast in the same as a notional camera lens viewing the three dimensional volume from the selected orientation. The pixel value is generated based on the voxels that are intercepted by the ray as it passes through the three dimensional volume. The manner in which the pixel value is assigned depends on the particular type of projection image that is used.

The projection localizer images 39 can be of further divided into various types according to the manner in which pixel values are assigned. Examples of possible types of projection images include mean intensity projection images, minimum intensity projection images, and maximum intensity projection images. However, it is not intended to exclude other types of projection images. In minimum intensity projection images, each pixel is assigned the minimum value of each voxel that is intercepted by the ray during ray casting. Similarly, in mean intensity projection images, each pixel is assigned a mean value of each voxel that is intercepted by the ray during ray casting. In maximum intensity projection images, each pixel is assigned the maximum value of each voxel that is intercepted by the ray during ray casting.

Referring again made to FIG. 8, at step (812), the pixel value generated at step (810) is stored.

At step (814), localizer image generation module 18 determines whether or not all pixels in the localizer image 39 have been assigned a value. If yes, then the process ends at step (816). If not, then step (806) is repeated.

It should be understood that FIG. 8 is intended to be exemplary only. The projection localizer images 39 may be created in any appropriate manner. In particular, a variety of methods can be utilized for creating projection images from a three dimensional volume including image based and object based methods. Thus, it is not intended to exclude any of these techniques.

While the various exemplary embodiments of the localizer display system 10 have been described in the context of medical image management in order to provide an application-specific illustration, it should be understood that localizer display system 10 could also be adapted to any other type of image or document display system.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative of the invention and non-limiting and it will be understood by persons skilled in the art that other variants and modifications may be made without departing from the scope of the invention as defined in the claims appended hereto.

1. A method of dynamically generating a localizer image comprising a plurality of pixel values, the method comprising:
(a) providing a plurality of planar images, wherein each image is associated with a plane;
(b) generating a three dimensional volume based on the plurality of planar images, wherein the three dimensional volume comprises a plurality of values;
(c) selecting an orientation and location for the localizer image; and,
(d) generating a localizer image in the selected orientation and location based on the plurality of values of the three dimensional volume.

2. The method as defined in claim 1, wherein the plurality of planar images comprises a plurality of medical study images that are substantially aligned.

3. The method as defined in claim 1, wherein the localizer image is a projection localizer image; and wherein (c) comprises selecting an orientation and a location relative to the three dimensional volume for the localizer image, and (d) comprises generating a projection localizer image in the selected orientation and location.

4. The method as defined in claim 1, wherein the localizer image is a planar localizer image; and wherein (c) comprises selecting a plane that passes through the three dimensional volume for the localizer image, and (d) comprises generating a planar localizer image in the selected plane.

5. The method as defined in claim 4, wherein the planar localizer image is an MPR image.

6. The method as defined in claim 3, wherein (d) comprises generating pixel values for the projection localizer image based on the voxel values of the three dimensional volume.

7. The method as defined in claim 6, wherein the projection localizer image is selected from one of a minimum intensity projection image, a mean intensity projection image, and a maximum intensity projection image.

8. The method as defined in claim 4, wherein (d) comprises generating a pixel value of the planar localizer image by interpolating a portion of the plurality of values of the three dimensional volume.

9. The method as defined in claim 8, wherein each of the values of the plurality of values comprises a voxel; and wherein (d) comprises generating at least one pixel value of the planar localizer image by
determining a position within the selected plane relative to the three dimensional volume corresponding to the at least one pixel; and,
tri-linearly interpolating a set of eight voxels of the three dimensional volume closest to the position corresponding to the at least one pixel.

10. A computer-readable medium upon which a plurality of instructions are stored, the instructions for performing the steps of the method as claimed in claim 1.

11. A system for dynamically generating a localizer image comprising a plurality of pixel values, the system comprising:
(a) a memory for storing a plurality of planar images, wherein each image has a plane;
(b) a processor coupled to the memory, said processor configured for:
(i) generating a three dimensional volume based on the plurality of planar images, wherein the three dimensional volume comprises a plurality of values;
(ii) selecting an orientation and location for the localizer image; and,
(ii) generating a localizer image in the selected orientation and location based on the plurality of values of the three dimensional volume.

12. The system as defined in claim 11, wherein the plurality of planar images comprises a plurality of medical study images that are substantially aligned.

13. The system as defined in claim 11, wherein the localizer image is a projection localizer image; and wherein (ii) comprises selecting an orientation and a location relative to the three dimensional volume for the localizer image, and (iii) comprises generating a projection localizer image in the selected orientation and location.

14. The system as defined in claim 11, wherein the localizer image is a planar localizer image; and wherein (ii) comprises selecting a plane that passes through the three dimensional volume for the localizer image, and (iii) comprises generating a planar localizer image in the selected plane.

15. The system as defined in claim 14, wherein the planar localizer image is an MPR image.

16. The system as defined in claim 13, wherein (iii) comprises generating pixel values for the projection localizer image based on the voxel values of the three dimensional volume.

17. The system as defined in claim 16, wherein the projection localizer image is selected from one of a minimum intensity projection image, a mean intensity projection image, and a maximum intensity projection image.

18. The system as defined in claim 14, wherein (iii) comprises generating a pixel value of the planar localizer image by interpolating a portion of the plurality of values of the three dimensional volume.

19. The system as defined in claim 18, wherein each of the values of the plurality of values comprises a voxel; and wherein (iii) comprises generating at least one pixel value of the planar localizer image by:
determining a position within the selected plane relative to the three dimensional volume corresponding to the at least one pixel; and,
tri-linearly interpolating a set of eight voxels of the three dimensional volume closest to the position corresponding to the at least one pixel.
